(19) United States
(12) Patent Application Publication
(10) Pub. No.: US 2007/0299134 A1
(43) Pub. Date: Dec. 27, 2007

(54) Concentrated and odorless omega 3 fatty acids

(76) Inventors: Larry Eugene Miller, Cincinnati, OH (US); Ashok Permchand Luhadiya, Cicinnati, OH (US); Haile Mehandho, Hamilton, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/821,224

(22) Filed: Jun. 22, 2007

Related U.S. Application Data
(60) Provisional application No. 60/815,991, filed on Jun. 23, 2006.

(51) Int. Cl. A61K 31/22; C07C 51/43

(52) U.S. Cl. 514/547; 554/174

(57) ABSTRACT

A blend of triglycerides having the general formula CH₂(OOC-R1)—CH(OOC—R2)—CH₂(OOC—R3), wherein at least about 50% of the R1, R2 and R3 groups have a chain length of C19 or more. The blend of triglycerides has less than about 50 parts per billion (ppb) of trimethyl amine, that is, they are substantially odor free. Also provided is a process for preparing an enriched blend of triglycerides by taking an oil comprising a mixture of polyunsaturated fatty acids and reacting the oil with a lower alcohol to form a mixture of esters. The mixture of esters is then distilled to remove a portion of the esters having a chain length of about C18 and below to form an enriched stream of enriched esters having at least about 50% of the esters with chain length of about C20 or more. The enriched stream of esters is then reacted with glycerine to form a triglyceride mixture, wherein the triglyceride mixture has less than about 10%, by weight of diglycerides and less than about 5%, by weight monoglycerides. The triglyceride mixture preferably comprises less than 5% diglyceride and less than 1% monoglyceride. The triglyceride mixture most preferably comprises less than 2.5% diglyceride and less than 0.2% monoglyceride. Finally, the triglyceride mixture may optionally be treated by water washing, bleaching, evaporation, steam stripping and mixtures of these, to remove residual free fatty acids, aldehydes, color bodies, odor bodies, peroxides and esters.

### CROSS REFERENCED TO RELATED APPLICATIONS

This application claims the benefit of U.S. Provisional Application No. 60/815,991, filed Jun. 23, 2006.

### TECHNICAL FIELD

This disclosure relates to triglyceride compositions made from fish oil, vegetable oil or any other oil containing Omega-3 fatty acids. The present triglyceride compositions are rich in Omega-3 fatty acids and are light colored or colorless and have no off flavors or offensive odor. Methods of making these triglyceride compositions are also provided.

### BACKGROUND OF THE INVENTION

Alpha linolenic acid (C18:3; (9Z,12Z,15Z)-Octadeca-9,12,15-trienoic acid, “ALA”), eicosapentaenoic acid (C20:5; (5Z,8Z,11Z,14Z,17Z)-icosa-5,8,11,14,17-pentaenoic acid, “EPA”), and docosahexaenoic acid (C22:6; (4Z,7Z,10Z,13Z,16Z,19Z)-docosa-4,7,10,13,16,19-hexaenoic acid, “DHA”) are long chain polyunsaturated fatty acids having multiple non-conjugated carbon-carbon double bonds with the first of their double bonds at the third carbon from the methyl terminus of the fatty acid and are often collectively referred to as “omega-3 fatty acids” or simply “omega-3s”. Other common omega-3 fatty acids include, but are not limited to, stearidonic acid (C18:4), eicosatetraenoic acid (C20:4), and docosapentaenoic acid (C22:5). These omega-3 fatty acids are known to have anti-inflammatory functions (enhancing immune response), are effective in the prevention of and therapy for certain thrombotic maladies, for controlling the content of triglycerides in blood in a living system, and for preventing certain thrombotic disturbances (such as, for example, heart attacks, strokes, and the like). Numerous clinical studies have found that omega-3s may further benefit patients with rheumatoid arthritis, high blood pressure, neurodermatitis, and certain other disorders. In response in part to these clinical results, many international institutions and authorities now recommend that individuals increase their daily consumption of omega-3 fatty acids and other polyunsaturated fatty acids (“PUFAs”).

Edible oils, such as fish oil and vegetable oils, are composed of triglycerides. Triglycerides are esters of glycerol with three long chain carboxylic acids (“fatty acids”). In the omega-3 fish oils and vegetable oils, a portion of the triglycerides in the oil include at least one ester of an omega-3 fatty acid. Typically, omega-3 fatty acids are consumed from two sources, in the daily diet and/or as dietary supplements. The primary source of omega-3 fatty acid in the diet is from fish oil and/or vegetables oils rich in omega-3 fatty acids. However, most people do not consume enough fish and/or vegetables rich in omega-3 fatty acids to achieve the recommended levels of consumption of omega-3s. As such, dietary supplements may be necessary for certain people to achieve the health benefits associated with omega-3 fatty acids.

Fish oils contain varying amounts of omega-3 fatty acids, depending several factors including the type of fish. For example, salmon oil may contain EPA at up to 18% by weight of total fatty acid (“TFA”), and DHA at 12% by weight of TFA. In general, however, the concentration of desirable omega-3 fatty acids is low in fish oils and the amount of fish oil consumed by an average person through normal diet is typically low. While there are natural limits to highly concentrated PUFA triglycerides from fish oils, on account of the triglyceride composition, the typical total content of EPA and DHA in fish oils is approximately 10-25% by weight of TFA.

The fish oils containing omega-3 fatty acids can be obtained as by-products in the production of products such as low-fat fish meal and fish cakes and from oil expression by methods such as boiling or expressing methods. Omega-3 containing fish oil may be obtained from a variety of fish, such as, but not limited to, sardine and/or pilchard, chub mackerel, pacific saury, Alaskan pollack, cod, anchovies, herring, salmon, tuna, and the like. The oil-expressing method employed in obtaining fish oils may be crude and commonly invites lowering of freshness of the material before oil-expression and formation of low-molecular, volatile amines, which are unpleasant smelling substances (e. g. trimethylamine, dimethylamine, and ammonia). Trimethylamine (“TMA”) is one of the major volatile amine compounds associated with the typical “fishy” odor. It is produced by an enzymatic conversion of trimethylamine oxide (“TMAO”), which is an osmo-regulatory compound in many marine fish. During the extraction and storage, the generation and mingling of these unpleasant smelling volatile amines in the fish oil cannot be avoided.

Fish oil also contains amounts of smaller chain length fatty acids, and other highly unsaturated fatty acids in addition to the omega-3s. The double bonds in the fatty acid chains of the omega-3s and other PUPAs in fish oils are susceptible to oxidation by oxygen and other oxidizing agents. The spoilage of fish oil by oxidation and/or bacterial action during storage may result in low molecular weight acids and low molecular weight compounds, such as ketones and aldehydes, in the oil producing undesirable colors, flavors, and/or odors in the oil. Therefore, even though fish oil freshly expressed from natural materials may have no perceptible odor, the production of low molecular amines (TMA) and oxidation products, such as ketones and aldehydes, during storage may give the oil an undesired color, flavor, and/or odor, thereby lowering of the commercial value of the fish oil.

In order to prevent emission of such fish-oil-odors, conventional methods may subject fish oil to refining treatments such as deacidification, deodorization and the like to remove impurities. However, even though conventional refining methods may remove some of the odor causing compound, it may still be impossible to remove completely the volatile amines, aldehydes and ketones since these compounds result from further degradation of the oil or components within the oil upon storage. In addition, there is a tendency in refined or concentrated oils for the emission of fishy odors to become more significant, since the refined fish oil contains higher concentrations of highly unsaturated fatty acids, such as EPA and DHA. Further, trimethylamine and other volatile amine compounds have very low odor threshold values (i.e., very low concentrations of TMA and other volatile amines are readily detected by the human sense of smell). When such oils produced by various commercial methods are incorporated into beverages or food products, the beverages or food products have a noticeable fishy taste and/or smell which many consumers find undesirable.

By using biocatalytic methods (employing lipases) it is possible to increase the Omega-3 fatty acid concentration in triglycerides to the range around DHA 40% by weight of TFA (Marinol D40.RTM. from Loders & Crocklaan, Wormerveer, Netherlands). However, natural oils cannot be produced in this manner.

Producing relatively highly enriched Omega-3 fatty acid concentrates (EPA+DHA>30% by weight. of TFA) in the form of natural triglycerides is therefore currently a challenge which has not been met (Haraldsson, G., G. (2000): Enrichement of Lipids with EPA and DHA by lipase. In Enzymes in Lipid Modification. Ed. U. Bornscheuer, Wiley VCH). Common distillation processes can, in theory, be used to be used to remove lower chain length fatty acids from fish oils, or other oil containing Omega-3 fatty acids. But the harsh conditions of the distillation process results in substantial degradation of the desired Omega-3 fatty acids. As such, the desired Omega-3 fatty acids are ultimately degraded and both streams leaving the distillation column are of less commercial value than the crude oil fed into the column.

Likewise, complex chromatographic methods or short-path distillation can be used to produce triglyceride fractions which are highly enriched with Omega-3 fatty, in theory. But many problems occur (W. M. Willis, R. W. Lencki and A. G. Marangoni (1998): Lipid modification strategies in the production of nutritionally functional fats and oils. Crit. Rev. Food Sci., 38(8), 639-674, Hayashi, K. and H. Kishimira (1996): Preparation and purification of DHA-enriched triacylglycerols from fish oils by column chromatography. Fisheries Science, 62(5), 842-843). These methods are expensive and complex. Furthermore, a thermal load of the Omega-3 fatty acids which are labile to oxidation is undesirable and leads to product decomposition. Generally, these methods therefore lead to oils which are no longer natural.

Often, to produce Omega-3 fatty acid concentrates, it is not natural oils which are used, but rather fatty acid or ester mixtures which are enriched, for example, by urea precipitation (S. P. J. N. Senanyake and F. Shahidi (2000): Concentration of docosahexaenoic acid (DHA) from algael oil via urea complexation. J. of Food Lipids, 7, 51-61, W. M. N. Ratnayake, B. Olsson, D. Matthews and R. G. Ackmann (1988): Preparation of omega-3 fatty acids from fish oil via urea complexation. Fat. Sci. Technol., 10, 381-386). In these cases, very pure Omega-3 fatty acid concentrates may likewise be produced. However, precipitation with urea is not suitable for triglycerides. Furthermore, the FDA has reported a physiologically hazardous formation of carbamate (carcinogenic class of substances) in the precipitation with urea (B. J. Canas (1999): Ethyl carbamate formation during urea complexation, for fractionation of fatty acids. JAOCS, 76(4), 537).

Only by using microorganisms is it possible to produce natural PUFA oils which have higher concentrations of PUFAs than fish oils or plant oils (K. D. Mukherjee (1999): Production and use of microbial oils. Inform, 10(4), 308-313). For instance, these oils can contain DHA at up to 40% by weight of TFA, GLA at 30% by weight of TFA, or ARA at 40% by weight of TFA. But these processes are prohibitively expensive.

As such, there exists a need for a concentrated mixture of Omega-3 fatty acids that is odorless and does not have a bad flavor. Moreover, there exists a need for a method of processing commercially available and relatively inexpensive sources of crude fish and vegetable to produce an enriched and concentrated stream of Omega-3 fatty acids. These and other advantages over prior compositions and processes are provided by the present invention.

### SUMMARY OF THE INVENTION

The present invention provides a blend of triglycerides having the general formula CH₂(OOC—R1)—CH(OOC—R2)—CH₂(OOC—R3), wherein at least about 50% of the R1, R2 and R3 groups have a chain length of C19 or more. Preferably at least about 60%, more preferably at least about 70%, and even more preferably at least about 80%, of the R1, R2 and R3 groups have a chain length of C19 or more. It is also preferred that the blend of triglycerides have less than about 50 parts per billion (ppb) of trimethyl amine, that is, they are substantially odor free.

In yet another aspect of the present invention there is provided a process for preparing an enriched blend of triglycerides comprising the steps of: taking an oil comprising a mixture of polyunsaturated fatty acids and reacting the oil with a lower alcohol to form a mixture of esters. The mixture of esters is then distilled to remove a portion of the esters having a chain length of about C18 and below to form an enriched stream of enriched esters having at least about 50% of the esters with chain length of about C20 or more. The enriched stream of esters is then reacted with glycerine to form a triglyceride mixture, wherein the triglyceride mixture comprises less than about 10%, by weight of diglycerides and less than about 5%, by weight monoglycerides. The triglyceride mixture preferably comprises less than 5% diglyceride and less than 1% monoglyceride. The triglyceride mixture most preferably comprises less than 2.5% diglyceride and less than 0.2% monoglyceride. Finally, the triglyceride mixture may optionally be treated by water washing, bleaching, evaporation, steam stripping and mixtures of these, to remove residual free fatty acids, color bodies, odor bodies peroxides and esters. This process preferably produces a mixture of triglycerides having the general formula CH₂(OOC—R1)—CH(OOC—R2)—CH₂(OOC—R3), wherein at least about 50% of the R1, R2 and R3 groups have a chain length of C19 or more. And even more preferably, the mixture of triglycerides has less than about 50 parts per billion (ppb) of trimethyl amine.

The triglyceride compositions of this invention can be made from relatively inexpensive and abundant sources of oil, such as fish oil, vegetable oil or any other oil containing Omega-3 fatty acids. The resulting triglyceride compositions are rich in Omega-3 fatty acids and are light yellow in color, slight to no fishy flavor, and have no off flavors. The concentrated mixture of Omega-3 fatty acids are light yellow in color, has a mild to slight fishy odor, and has an excellent flavor profile.

### DETAILED DESCRIPTION OF THE INVENTION

As used herein, the term “comprising” means various components conjointly employed in the preparation of the triglyceride compositions of the present invention. Accordingly, the terms consisting essentially of” and “consisting of” are embodied in the term “comprising”. As used herein, all parts, percentages and ratios are based on weight unless otherwise specified.

Oil Processing

While the present invention is explained and exemplified with the processing of fish oils to produce concentrated Omega-3 fatty acids, those skilled in the art will appreciate that the processes described herein can be used with any oil containing PUFAs. Fish oil, however, is an abundant source of PUFAs and Omega-3 fatty acids, and it presents the additional challenge of possessing bad smell, bad taste, and off color.

The fish oils to be used as the starting materials in the present invention include not only the fat and oils obtained from such fish as sardine and/or pilchard, chub mackerel, pacific saury and the like, expressed according to a conventional method, but the fat and oils removed from viscera of pollack, shark, etc. and also from such Mollusca as squid and/or cuttle fish, octopus, etc.

The fish oil used as the starting material in the present invention may be crude fish oil expressed from fish, but in order to improve the efficiency of deodorization and molecular distillation that are to be operated in the later stages, it is desirable that the crude fish oil is subjected to acid refinement by means of phosphoric acid, sulfuric acid and the like, or to alkali treatment by means of caustic alkali, then further to the preliminary refinement such as deacidification, decoloration, dewaxing, etc. to obtain the product having higher content of triglycerides. Particularly for maintaining a transparent liquid state that does not cloud at normal temperature

The crude fish oil can be pre-treated to clean and purify it. Those skilled in the art will appreciate that amount and necessity of pre-processing will depend on the quality of the crude oil stream. Standard cleaning steps know to skilled artisans can be used alone or in combination. For example, winterization, filtration, adsorption, evaporation and steam stripping are all methods that can be used depending on the needs of the crude fish oil stream. More specifically, the fish oil stream can be optionally filtered to remove solids. The filtered oil may then be fed into an adsorption column. Color and odor bodies are removed and then filtered and caked. Moreover, the fish oil stream can be optionally treated with steam to strip out free fatty acids and other volatile species.

After pre-treating, the fish oil is converted to esters of fatty acids in a common reaction vessel. The fish oil is treated with a lower alcohol, for example methanol or ethanol, in the presence of a catalyst. Any of a variety of common catalysts can be used, for example, sodium methoxide or potassium carbonate. Glycerine is the major by-product of the reaction of triglyceride oils with lower alcohols. Most of the glycerine is removed and the esterified oil is then moved to a different vessel.

After the glycerine is removed, the esterified oil can optionally be flashed to remove excess lower alcohol, for example, methanol, which can be recycled if desired. After the optional flashing the esterified oil is preferably washed with water to remove water-soluble bodies, for example, glycerine, soap, lower alcohols, odor bodies, color bodies and flavor bodies. The washed, esterified oil is then dried to remove residual water and residual water-soluble bodies. Those skilled in the art will appreciate that flashing, water washing and drying are common processes, as are the treatment vessels themselves.

The next step is fractional distillation wherein lower chain length esters and other residual low boilers, for example, lower alkyl esters (methyl and ethyl esters, for example) are removed. It is preferred, that the C18 and lower esters are removed during distillation to increase the concentration of C20, C22 and longer esterified fatty acids. While C18, linolenic acid is generally considered one of the beneficial Omega-3 fatty acids, it is preferred to increase the concentration of the longer C20 and C22, EPA and DHA. By targeting the distillation process to concentrate the longer esterified fatty acids, some of the C18 fatty chains such as linolenic acid or methyl linoleate will, of course, be retained, but the overall concentration of Omega-3 fatty acids, and specifically, EPA and DHA will be maximized in the final product.

Optionally, after distillation, a wiped film evaporator can be used to further purify the longer chain ester stream by removing some of the high boiling residual compounds such as sterols including cholesterol and sterol esters including cholesterol esters.

Having removed both high boiling residuals and low boiling residuals, the middle cut of purified and concentrated esters is then moved to a reaction vessel. Glycerin is added to this reactor to reconvert the esters to triglycerides. Preferably, the glycerin is added to the reactor such that the esters are in stoichiometric excess in the reactor. By stoichiometric excess it is meant that the molar ratio of ester to glycerol is at least about 3:1, preferably at least about 4:1, and more preferably at least about 5:1. This reaction is conducted in the presence of a catalyst, for example, potassium carbonate or sodium methoxide. The desired product has about 90%, by weight, triglycerides. The maximum desired concentration of monoglycerides is about 5% or less, and the maximum desired concentration for diglycerides is about 10% or less. The triglyceride mixture preferably comprises greater than 90% triglyceride, less than 5% diglyceride and less than 1% monoglyceride. The triglyceride mixture most preferably comprises greater than 95% triglyceride, less than 2.5% diglyceride and less than 0.2% monoglyceride.

After the product stream is converted to triglycerides, purification and concentration steps follow. It is preferred that the product stream move first to a water wash, which is even more preferably conducted in two stages. Potassium citrate can be added to convert the catalyst to a more easily removed compound, for example, potassium carbonate. Free fatty acids are converted to soap which are easily removed with water. After the water wash the undesired components are discarded and the triglyceride product stream is dried using a conventional dryer.

Following the removal of moisture, the triglyceride product stream is bleached. Bleaching is preferably carried out in a column with silica gel, bleaching earth, alumina or the like. Color bodies, flavor bodies, odor bodies and oxidized species, such as peroxides that are often precursors to off flavor and off odor development, if present, are removed. The triglyceride product moves from the bleaching process to an evaporation column where residual esters and any remaining free fatty acids are removed. A final, and optional step, is steam stripping. Steam can remove small amounts of the color bodies, flavor bodies, odor bodies, peroxide, if present, and free fatty acids. The cleaning and stripping steps described above can be used individually or in combination to achieve the desired purity for the final product.

At this stage the triglyceride mixture should be substantially free of odor bodies and have no off flavors. As used herein, the terms “substantially free of odor bodies” means that the triglyceride mixture has less than about 50 ppb trimethyl amine.

After the triglyceride mixture is cleaned and stripped, preservatives, antioxidants and the like can be added to preserve the product.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A blend of triglycerides having the general formula CH₂(OOC—R1)—CH(OOC—R2)—CH₂(OOC—R3), wherein at least about 50% of the R1, R2 and R3 groups have a chain length of C19 or more.

2. The blend of triglycerides of claim 1, wherein at least about 60%, preferably at least about 70%, and more preferably at least about 80%, of the R1, R2 and R3 groups have a chain length of C19 or more.

3. The blend of triglycerides of claim 1 wherein the triglyceride mixture comprises greater than about 90% triglyceride, less than 5% diglyceride and less than about 1% monoglyceride.

4. The blend of triglycerides of claim 1 which has less than about 50 parts per billion (ppb) of trimethyl amine.

5. The blend of-triglycerides of claim 1 which has less than about 1000 ppm lower alcohol esters.

6. A process for preparing an enriched blend of triglycerides comprising the steps of:
a) taking an oil comprising a mixture of polyunsaturated fatty acids and reacting the oil with a lower alcohol to form a mixture of esters;
b) distilling the mixture of esters to remove a portion of the esters having a chain length of about C18 and below to form an enriched stream of enriched esters having at least about 50% of the esters with chain length of about C20 or more;
c) reacting the enriched stream of esters with glycerine to form a triglyceride mixture, wherein the triglyceride mixture comprises less than about 10% by weight diglycerides and less than about 5% by weight monoglycerides; and
d) optionally treating the triglyceride mixture by water washing, bleaching, evaporation, steam stripping and mixtures of these, to remove residual free fatty acids, aldehydes, color bodies, odor bodies, peroxides and esters.

7. The process of claim 6 wherein the mixture of triglycerides has the general formula CH₂(OOC—R1)—CH(OOC—R2)—CH₂(OOC—R3), wherein at least about 50% of the R1, R2 and R3 groups have a chain length of C19 or more.

8. The process of claim 6 wherein the thriglyceride mixture comprises less than 5% diglycerides and lass than 1% monoglyceride.

9. The process of claim 6 wherein the mixture of triglycerides have less than about 50 parts per billion (ppb) of trimethyl amine.

10. The process of claim 6 wherein at least about 60%, preferably at least about 70%, and more preferably at least about 80%, of the R1, R2 and R3 groups have a chain length of C19 or more.

11. The process of claim 6 wherein the lower alcohol is methanol or ethanol.

12. The process of claim 6 wherein the residual free esters are removed to below about 1000 ppm.
